(19) United States
(12) Patent Application Publication
(10) Pub. No.: US 2023/0001108 A1
(43) Pub. Date: Jan. 5, 2023

(54) HANDS-FREE ELECTROLYTE DISPENSER

(71) Applicant: BOA Nutrition, Inc., Raleigh, NC (US)

(72) Inventors: Hank Durschlag, Raleigh, NC (US); Jon Pritchett, Raleigh, NC (US)

(21) Appl. No.: 17/839,158

(22) Filed: Jun. 13, 2022

Related U.S. Application Data
(60) Provisional application No. 63/209,819, filed on Jun. 11, 2021.

(51) Int. Cl. A61M 11/02

(52) U.S. Cl. CPC A61M 11/02; A61M 2202/0482; A61M 2210/0625; A61M 2209/082

(57) ABSTRACT

A system is described for the hands-free administration of a nutritional supplement matrix to the oral mucosa. A two-piece mouthpiece is formed in a nozzle cap that accepts canisters with the nutritional supplement matrix under pressure. Bite pressure administered by a user to an upper moveable portion of the two-piece mouthpiece may activate the dispensing of an atomized mixture of the nutritional supplement matrix. A flexible joiner between the upper moveable portion and the lower fixed portion of the two-piece mouthpiece may compress, fold, or otherwise collapse under the bite pressure from the user.

The present application claims priority to U.S. Provisional Patent Application 63/209,819 filed Jun. 11, 2021, which is incorporated by reference.

### FIELD OF THE INVENTION

The present invention is generally related to the delivery of nutritional supplements through the oral mucosa, specifically the storage and delivery devices needed to deliver the nutritional supplements.

### BACKGROUND

Excessive fluid loss through perspiration during intense exercise may leave an athlete with an electrolyte deficiency in addition to dehydration. Low levels of electrolytes in the system can lead to cramping. Salt pills provide large salt doses to attempt to overcome this issue with endurance athletes. This approach can oversupply the body with sodium to overcome the delay in bioavailability through the GI tract. A mechanism for delivering a smaller dose of electrolytes to the buccal mucosa may provide the necessary electrolytes with more rapid bioavailability.

During exercise, the body requires more oxygen than when the body is at rest. When oxygen cannot be delivered to the muscles faster to replace the used oxygen, the muscles will begin to convert the available glucose to lactic acid. This will build up in the muscle, causing fatigue. More rapid delivery of oxygen to the system during exertion can reduce fatigue. Supplemental oxygen therapy has been demonstrated to improve athletic performance and decrease reaction times.

The benefits of proper hydration and supplemental oxygen therapy on athletic performance are demonstrable. Therefore, it is desirable to have a means of delivering electrolytes and oxygen to an athlete during athletic activities that do not impede on those activities.

### BRIEF DESCRIPTIONS OF THE DRAWINGS

FIG. 1 illustrates a hands-free electrolyte dispenser.

FIG. 2 illustrates a flexible joiner.

FIG. 3 illustrates a bicycle attachment.

### SUMMARY

A hands-free electrolyte dispenser is described. The dispenser delivers an aerosol of an electrolytic composition. The dispenser includes a nozzle affixed or connected to a canister or supply holding the electrolytic composition. The nozzle includes a mouthpiece that is activated by a user biting on the mouthpiece. A user may activate the mouthpiece without using their hands.

In one aspect, a system for hands-free delivery of an aerosol composition to an oral mucosa is described. The system includes a canister containing a nutritional supplement composition under pressure. A nozzle is affixed or connected to the canister. The nozzle comprises a two-piece mouthpiece. The system is configured to dispense an aerosol of the composition when a bite pressure is applied to the two-piece mouthpiece.

In another aspect, a system for hands-free delivery of an aerosol composition to an oral mucosa is described. The system includes a canister containing a nutritional supplement composition under pressure. A nozzle is affixed or connected to the canister. The nozzle includes a moveable upper portion and a fixed lower portion. The system is configured to dispense an aerosol of the composition when a downward pressure is applied to the moveable upper portion.

In another aspect, a nozzle for hands-free delivery of an aerosol of a composition to a buccal mucosa is described. The nozzle includes a moveable upper portion. The nozzle includes a fixed lower portion. The moveable upper portion and the fixed lower portion form a mouth portion. The nozzle is configured to dispense an aerosol of a composition from the mouth portion to the buccal mucosa when a pressure is applied to the moveable upper portion in a direction towards to the fixed lower portion.

### DETAILED DESCRIPTION

For purposes of this application, any terms that describe relative position (e.g., “upper”, “middle”, “lower”, “outer”, “inner”, “above”, “below”, “bottom”, “top”, etc.) refer to an aspect of the invention as illustrated, but those terms do not limit the orientation in which the aspect can be used.

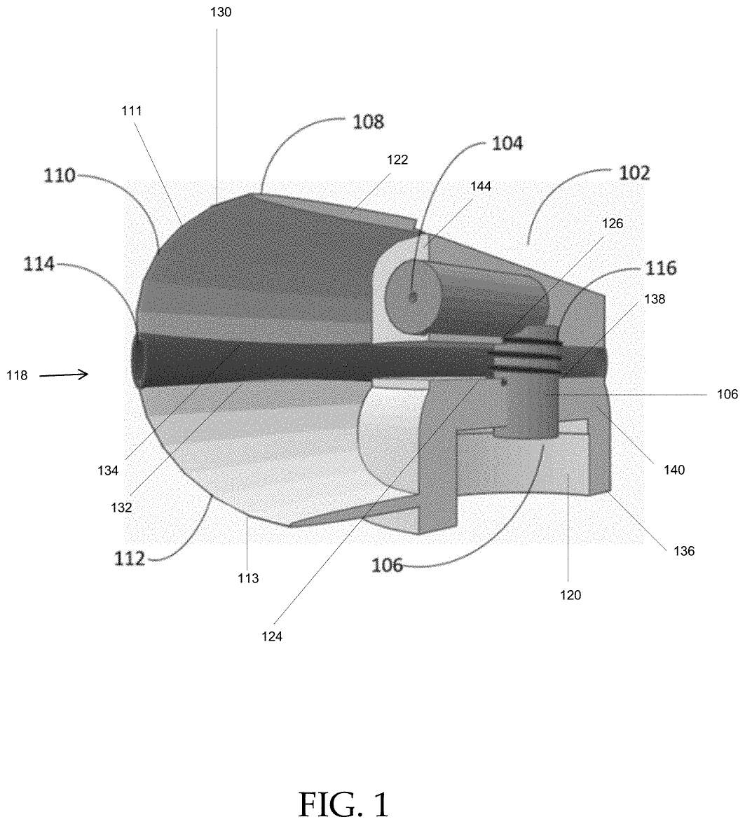

FIG. 1 illustrates a hands-free electrolyte dispenser system 100. The system 100 may comprise a nozzle cap 102, which may be fixed to a canister 304 holding a nutritional supplement matrix to enable transmucosal delivery of the nutritional supplement matrix. Transmucosal delivery of nutrient supplements offers advantages over oral delivery when negative issues relating to the gastrointestinal tract, the stomach, substance digestion and absorption, swallowing, protocol compliance, and substance effectiveness, and other issues to gastrointestinal metabolism are considered. The formulation or composition may include a (i) nutritional supplement matrix fraction, (ii) a gas fraction, (iii) an enhancer fraction, (iv) a liquid fraction, and (v) a preservative fraction wherein the nutritional supplement matrix fraction, the gas fraction, the enhancer fraction, the liquid fraction, and the preservation fraction are all mixed or combined and treated to maintain a state of balanced suspension among the oxygen molecules for a specific duration of time before being dispensed from a canister 304. Furthermore, the composition may be in a compressed state in the canister 304 before being dispensed. The nozzle cap 102 may be affixed or engaged to the canister 304, with its contents under pressure, to deliver an atomized aerosol spray of the contents. Such canisters 304 are well known in the art.

The nozzle cap 102 includes an outlet 104 that is configured for various spray patterns. The nozzle cap 102 may have a female member 106 that may connect to a male outlet of the canister 304. The female member 106 includes a lower cavity 120 in fluidic communication with the outlet 104. The female member 106 may include a duct or passage that connects with the outlet 104. In one aspect, the contents of the canister 304 may be released when the nozzle cap 102 is depressed downward. In one aspect, the contents of the canister 304 may be released when the nozzle cap 102 is moved to the side or laterally, such as with a pressurized whipped cream canister.

The nozzle cap 102 may include a two-piece mouthpiece 108 that allows the user to dispense contents through the nozzle cap 102 by biting down on the two-piece mouthpiece 108. In one aspect, the two-piece mouthpiece 108 includes of a moveable upper portion 110, a fixed lower portion 112, and a flexible joiner 114. The fixed lower portion 112 may rest on the canister 304 when the nozzle cap 102 is affixed to the canister 304. When the user bites down on the moveable upper portion 110, the nozzle cap 102 may prompt the release of an aerosol of the nutritional supplement from the canister 304. The moveable upper portion 110 is configured to move toward the fixed lower portion 112 to release the nutritional supplement from the canister 304. As such, when a sufficient pressure is applied to the moveable upper portion 110 in a direction towards to the fixed lower portion 112, the system 100 releases the nutritional supplement from the canister 304.

A combination of the fixed lower portion 112 and the moveable upper portion 110 define an opening 118 for the two-piece mouthpiece 108. In operation, a user may place their mouth around the opening 118 and bite down on an upper surface 122 of the moveable upper portion 110. The nutritional supplement matrix then passes from the canister 304, through the male member of the canister 304, to the female member 106, and to the outlet 104, where it is emitted in aerosol form.

The flexible joiner 114 may include a silicon, rubber, plastic or other pliable/resilient connection or engagement between the moveable upper portion 110 and the fixed lower portion 112 of the two-piece mouthpiece 108. The flexible joiner 114 allows the moveable upper portion 110 to move while maintaining a seal between the portions 110 and 112 to focus the release of the aerosol of the atomized nutritional supplement matrix towards the buccal mucosa. The flexible joiner 114 may compress in height or shape when then the user bites down on the moveable upper portion 110. The flexible joiner 114 may extend most of or all of a junction between the moveable upper portion 110 and the fixed lower portion 112. The flexible joiner 114 may engage an upper edge of the 132 of the fixed lower portion 112 and a lower edge 134 of the moveable upper portion 110.

In certain aspects, the two-piece mouthpiece 108 includes a reset spring 116 between the moveable upper portion 110 and fixed lower portion 112 of the two-piece mouthpiece 108, which may return the moveable upper portion 110 to its resting position. The reset spring 116 is positioned between the moveable upper portion 110 and the fixed lower portion 112 to urge the moveable upper portion away from the fixed lower portion 112. This may allow for easy hands-free operation of the nozzle cap 102. In some embodiments, the reset spring 116 assists in stopping the delivery of the nutritional supplement matrix, as the reset spring 116 lifts the female member 106 from the male member of the canister 304.

In certain embodiments, the female member 106 may be positioned in a central opening of the reset spring 116. A lower side of the reset spring 116 may bias against an upper surface 124 of the fixed lower portion 112, and an upper side of the reset spring 116 may bias against a lower surface 126 of the moveable upper portion 110. In this aspect, the reset spring 116 includes a coil spring. In other aspects, other biasing members may be used to bias the moveable upper portion 110 away from the fixed lower portion 112.

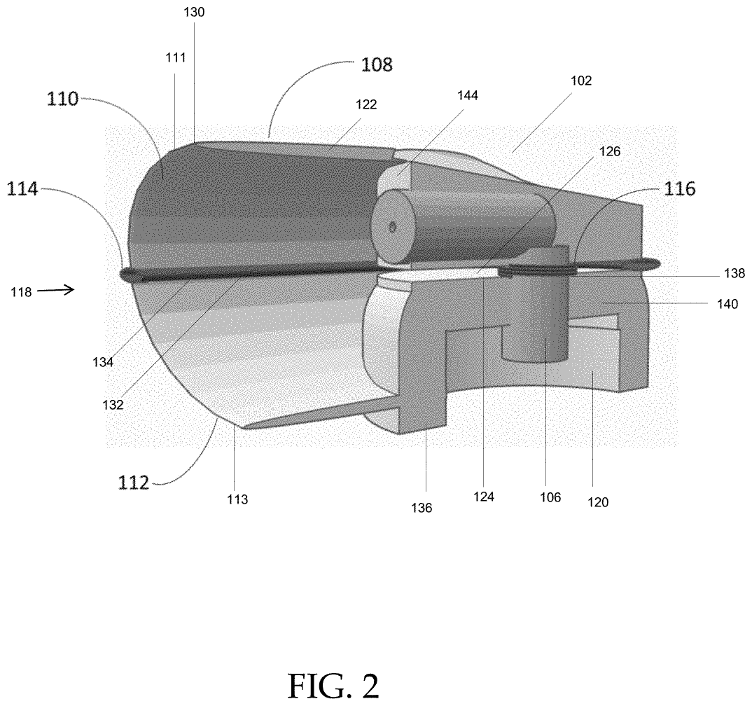

The fixed lower portion 112 and the moveable upper portion 110 combine to form a mouth portion 130. The outlet 104 may be positioned in the mouth portion 130. In the aspect of FIGS. 1 and 2, the mouth portion 130 defines the opening 118. The mouth portion 130 may extend from the nozzle cap 102. In the aspect of FIGS. 1 and 2, the moveable upper portion 110 includes an upper mouth portion 111, and the fixed lower portion 112 includes a lower mouth portion 113. The upper mouth portion 111 and the lower mouth portion 113 join to form the mouth portion 130. The upper mouth portion 111 and the lower mouth portion 113 may include complementary or semi-circular or arcuate shapes. The lower mouth portion 113 may extend from a base portion 140 of the fixed lower portion 112. The upper mouth portion 111 may extend from a lateral edge 144 of the moveable upper portion 110.

In the aspect of FIGS. 1 and 2, a lower rim 136 of the nozzle cap 102 connects, engages, or rests on the canister 304. The female member 106 fluidly receives the male member of the canister 304. In this aspect, the female member 106 is part of or integral with the moveable upper portion 110. In this aspect, the female member 106 passes through an opening 138 in the base portion 140 of the fixed lower portion 112. When the user bites or presses downward on the moveable upper portion 110, the moveable upper portion 110 moves downward relative to the fixed lower portion 112, the flexible joiner 114 is compressed, and the female member 106 moves downward in the opening 138 to actuate the male member of the canister 304 to dispense the nutritional supplement matrix. In certain aspects, the male member may be part of a pressure relief valve in the canister 304.

In some aspects, the nutritional supplement matrix may only be dispensed when the user bites down on the two-piece mouthpiece 108, and the dispensing ceases when the user releases the bite pressure. In one aspect, the nutritional supplement matrix may be dispensed in a metered-dose. In one aspect, the nutritional supplement matrix may be dispensed for the duration of the time bite pressure is applied to the two-piece mouthpiece 108. It should be obvious that there are numerous ways to shape the two-piece mouthpiece 108 so that it may comfortably be controlled and held in the user's mouth. For example, an aspect of the two-piece mouthpiece 108 may include an exterior that is molded to the user's mouth.

In other aspects, the moveable upper portion 110 may be movably engaged to the fixed lower portion 112. For example, the moveable upper portion 110 may be movably engaged to the fixed lower portion 112 via a hinge.

FIG. 2 illustrates an example of the flexible joiner 114 in a compressed state. In this aspect, when a bite pressure is applied to the moveable upper portion 110 of the two-piece mouthpiece 108, the flexible joiner 114 may compress, fold, or otherwise collapse to allow the nutritional supplement matrix to be dispensed from the system 100. In one aspect, the reset spring 116 may be compressed when the bite pressure is applied. The reset spring 116 may bias the moveable upper portion 110 to return to its resting position after the bite pressure is removed.

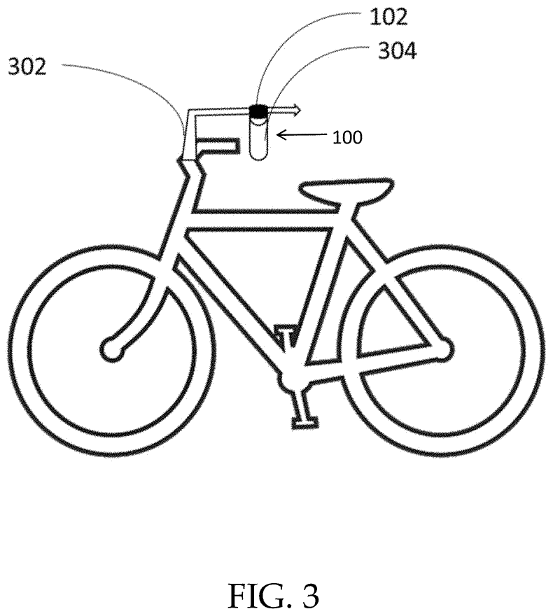

FIG. 3 illustrates a bicycle attachment 302 for the system 100. In this aspect, the nozzle cap 102 or the canister 304 may be joined to a bicycle by the bicycle attachment 302. For example, the bicycle attachment 302 may include a flexible arm that mounts to the handlebars and to the system 100. The nozzle cap 102 may be configured to accept the canister 304 that may include a nutritional supplement matrix. This may allow users to position the nozzle cap 102 near their mouths so they may take in the nutritional supplement matrix in a hand-free manner without interrupting their cycling.

In one aspect, a water reservoir is mounted to the bicycle frame, and a straw is held aloft by a mount on the handlebars to allow the cyclist to take in fluids hand-free. In one aspect, the nutritional supplement matrix delivery system 100 may be integrated with a hands-free hydration system. In one aspect, a mobile application may monitor a cyclist's intake of the nutritional supplement matrix, the distance traveled on the bicycle, and user physiological measurements to deliver recommendations on the proper timing and use of the system 100 and hydration needs. It should be obvious that this type of integration could be used on sporting equipment other than bicycles. For example, a similar system may be integrated with a treadmill. Hydration packs that may be worn as a backpack, around the waist, etc., and have a straw near the user's mouth known in the art. In one aspect, the nozzle cap 102 may be integrated with a hydration pack to deliver the same outlet's nutritional supplement matrix. For example, the canister 304 may be mounted to the shoulder strap of a backpack and allow the user to depress the same two-piece mouthpiece 108 to receive the nutritional supplement matrix and the additional hydration.

The canister 304 contains the nutritional supplement matrix which may include any one or more of (i) a nutritional supplement fraction, (ii) a gas fraction, (iii) an enhancer fraction, (iv) a liquid fraction and (v) a preservative fraction, wherein the enhancer fraction being a mucoadhesive to ensure adhesion of the atomized droplet to the membrane maximizing absorption. Furthermore, the particles of the composition can be positively or negatively charged so that they coalesce or disperse.

The supplement fraction may include a combination of electrolytes, vitamins, or minerals. Furthermore, the supplement fraction maybe selected from or be combination of Potassium chloride, Sodium chloride, Iron, Sodium, Calcium, Magnesium, Carbohydrates, Proteins, Zinc, Molybdenum, Caffeine, Copper, Potassium, Manganese, Chlorides, Bicarbonate and Carbonate, Aluminum, Arsenic, Bromine, Cadmium, Chromium, Sodium, Potassium, Chlorine, Cobalt, Fluorine, Iodine, Citicoline, Tyrosine Phenylalanine, Taurine, Malic Acid, Glucuronolactone, Manganese, Molybdenum Nickel, Phosphorus, Selenium, Silicon, Vanadium, Amino Acids, Vitamin A, Vitamin D, Vitamin E, Vitamin K, Vitamin C, Vitamin B complex, Thiamine (Vitamin 31), Riboflavin (Vitamin 132). Niacin (Vitamin B3), Pyridoxine (Vitamin B6), Biotin, Pantothenic Acid and Pantetheine, Folic Acid, Vitamin B12, “Unofficial” B Vitamins including Choline and Inositol, Vitamin P (bioflavonoids), and flavoring agents, and/or other vital nutrients, in addition to various homeopathic/alternative substances. In one embodiment the formulation or composition of nutritional supplement matrix comprises 50 to 60 weight % of Chloride, where the chloride is from potassium chloride and sodium chloride; 30 to 40 weight % of Sodium, where the sodium is from sodium chloride; and 5 to 15 weight % of Potassium where the potassium is from potassium chloride.

The gas fraction in the above-mentioned formulation or composition comprises ambient air, oxygen, or nitrogen, wherein the gas fraction maybe in a compressed state. In one embodiment the gas fraction is comprised of oxygen wherein the concentration of oxygen is up to 95% oxygen and can mixed with other gases such as nitrogen.

The enhancer fraction in the above-mentioned formulation or composition is a mucoadhesive enhancer, an absorption enhancer, or a flavoring. Wherein the mucoadhesive enhancer fraction is selected from a group of pectin's or apple pectin's. Wherein the absorption enhancer is selected from a group of glycerin's or vegetable glycerin's. Wherein the flavoring fraction is selected from a group of natural flavoring for foods and artificial flavoring for foods.

The liquid fraction in the above-mentioned formulation or composition is selected from a group of water, distilled water, filtered water, oxygenated water or saline and where the composition is water-soluble. Wherein the amount of water is sufficient to dissolve all elements of the composition and prevent any molecules from precipitating. Furthermore, wherein the liquid fraction can be a combination of liquids and used to adjust the pH of the composition to be close to a physiological pH.

The preservative fraction in the above-mentioned formulation or composition is selected from a group of food preservatives or potassium sorbate. The food preservative fraction helps maintain and extend the shelf life of the composition.

As such, it should be understood that the disclosure is not limited to the particular aspects described herein, but that various changes and modifications may be made without departing from the spirit and scope of this novel concept as defined by the following claims. Further, many other advantages of applicant's disclosure will be apparent to those skilled in the art from the above descriptions and the claims below.

What is claimed is:

1. A system for hands-free delivery of an aerosol composition to an oral mucosa, comprising;
a canister containing a nutritional supplement composition under pressure;
a nozzle is affixed or connected to the canister;
the nozzle comprises a two-piece mouthpiece; and
the system is configured to dispense an aerosol of the composition when a bite pressure is applied to the two-piece mouthpiece.

2. The system for hands-free delivery of an aerosol composition to the oral mucosa according to claim 1, wherein the two-piece mouthpiece comprises a moveable upper portion and a fixed lower portion.

3. The system for hands-free delivery of an aerosol composition to the oral mucosa according to claim 2, wherein the moveable upper portion is configured to move toward the fixed lower portion to release the aerosol composition from the canister.

4. The system for hands-free delivery of an aerosol composition to the oral mucosa according to claim 2, wherein the two-piece mouthpiece comprises a biasing member between the moveable upper portion and the fixed lower portion.

5. The system for hands-free delivery of an aerosol composition to the oral mucosa according to claim 2, wherein the moveable upper portion includes an upper mouth portion, and the fixed lower portion includes a lower mouth portion, and the upper mouth portion and the lower mouth portion form a mouth portion for the nozzle

6. The system for hands-free delivery of an aerosol composition to the oral mucosa according to claim 5, wherein a flexible joiner joins the upper mouth portion and the lower mouth portion.

7. The system for hands-free delivery of an aerosol composition to the oral mucosa according to claim 5, wherein the lower mouth portion extends from a base portion of the fixed lower portion, and the upper mouth portion extends from a lateral edge of the moveable upper portion.

8. The system for hands-free delivery of an aerosol composition to the oral mucosa according to claim 5, wherein the upper mouth portion and the lower mouth portion include complementary semi-circular or arcuate shapes.

9. The system for hands-free delivery of an aerosol composition to the oral mucosa according to claim 1, wherein the nozzle includes a female member configured to connect or engage to a male outlet of the canister.

10. The system for hands-free delivery of an aerosol composition to the oral mucosa according to claim 9, wherein the female member is part of or integral with a moveable upper portion, the female member passes through an opening in a fixed lower portion.

11. The system for hands-free delivery of an aerosol composition to the oral mucosa according to claim 10, wherein the female portion configured to move downward through the opening when a downward force is applied to the moveable upper portion and actuate the male member of the canister to dispense the nutritional supplement.

12. The system for hands-free delivery of an aerosol composition to the oral mucosa according to claim 11, wherein the female member is positioned in a central opening of a spring, a lower side of the spring biases against an upper surface of the fixed lower portion, and an upper side of the reset spring biases against a lower surface of the moveable upper portion.

13. A system for hands-free delivery of an aerosol composition to an oral mucosa, comprising;
a canister containing a nutritional supplement composition under pressure;
a nozzle is affixed or connected to the canister;
the nozzle comprises a moveable upper portion and a fixed lower portion; and
the system is configured to dispense an aerosol of the composition when a downward pressure is applied to the moveable upper portion.

14. The system for hands-free delivery of an aerosol composition to the oral mucosa according to claim 13, wherein a flexible joiner that connects a moveable upper portion and a fixed lower portion, and the flexible joiner configured to compress in height or shape when a downward force is applied to the moveable upper portion.

15. The system for hands-free delivery of an aerosol composition to the oral mucosa according to claim 14, wherein the flexible joiner engages an upper edge of the fixed lower portion and a lower edge of the moveable upper portion.

16. The system for hands-free delivery of an aerosol composition to the oral mucosa according to claim 14, wherein flexible the joiner extends most of or all of a junction between the moveable upper portion and the fixed lower portion.

17. The system for hands-free delivery of an aerosol composition to the oral mucosa according to claim 13, a reset spring is configured to bias the moveable upper portion away from the fixed lower portion.

18. A nozzle for hands-free delivery of an aerosol of a composition to a buccal mucosa, comprising:
a moveable upper portion;
a fixed lower portion;
the moveable upper portion and the fixed lower portion form a mouth portion;
and
the nozzle configured to dispense an aerosol of a composition from the mouth portion to the buccal mucosa when a pressure is applied to the moveable upper portion in a direction towards to the fixed lower portion.

19. The nozzle for hands-free delivery of an aerosol of a composition to the buccal mucosa according to claim 18, wherein the moveable upper portion moves relative to the fixed lower portion, and a biasing member is positioned between the moveable upper portion and the fixed lower portion to urge the moveable upper portion away from the fixed lower portion.

20. The nozzle for hands-free delivery of an aerosol of a composition to the buccal mucosa according to claim 18, wherein a flexible joiner connects a moveable upper portion and a fixed lower portion.
